(19) United States
(12) Patent Application Publication
(10) Pub. No.: US 2022/0280410 A1
(43) Pub. Date: Sep. 8, 2022

(54) COSMETIC COMPOSITION COMPRISING AN ORAL CAPSULE CONTAINING HYALURONIC ACID AND WHEAT GRAIN OIL WITH PHYTOCERAMIDES

(71) Applicant: IIAA LIMITED, Stanmore Middlesex (GB)

(72) Inventors: Lorraine PERRETTA, Stanmore Middlesex (GB); Paul NAUDE, Stanmore Middlesex (GB); Tanya MEYER, Stanmore Middlesex (GB); David ALPERT, Stanmore Middlesex (GB)

(73) Assignee: IIAA LIMITED, Stanmore Middlesex (GB)

(21) Appl. No.: 17/628,400

(22) PCT Filed: Jul. 22, 2020
(86) PCT No.: PCT/GB2020/051750

§ 371 Date: Jan. 19, 2022

(30) Foreign Application Priority Data
Jul. 22, 2019 (GB) 1910450.4

(51) Int. Cl. A61K 8/73; A61K 8/92; A61K 8/68; A61K 8/11; A61Q 19/08

(52) U.S. Cl. CPC A61K 8/735; A61K 8/922; A61K 8/68; A61K 8/11; A61Q 19/08; A61K 2800/92

(57) ABSTRACT

A capsule for oral administration, comprising: a filling having from 30 to 50% by weight hyaluronic acid; and from 55 to 65% by weight of a wheat grain extract oil comprising phytoceramides. The phytoceramides are present in the capsule in an amount of from 0.1 to 5% by weight. The % by weight of each component is measured relative to the total combined weight of the hyaluronic acid and wheat grain extract oil. Also related methods, uses and further products.

### FIELD OF THE INVENTION

The present invention relates generally to cosmetic compositions for use in improving the overall appearance of the skin, particularly by reducing signs of ageing, improving the hydration of the skin and/or reducing the severity of the retinoid reaction or preventing the retinoid reaction. More particularly, this invention concerns a capsule for oral administration, comprising hyaluronic acid and phytoceramides. The invention also concerns related methods, uses and further products.

### BACKGROUND OF THE INVENTION

Numerous skincare products have been developed for improving the appearance of human skin. In particular, products which help maintain the skin in a hydrated state are known to improve the appearance of the skin, for example to prevent or reverse signs of aging.

The topical application of lipid emollients can be used to protect, moisturize and lubricate the skin, supplementing the natural function of sebum produced by healthy skin. Such emollients act by providing an occlusive lipid coating on the surface of the epidermis, thereby blocking water evaporation from the skin surface. Additionally or alternatively, hygroscopic humectants may be used to attract and retain water molecules, thereby keeping the skin hydrated. Emollients are used widely in topical creams, particularly oily creams, in addition to ointments; humectants are also used in topical creams, and particularly in moisturizers, face cleansers, lip balms and eye creams. Humectants and emollients can be used in combination in topical creams, in order to both attract and seal in water molecules.

In addition to topically applied products, various orally administered compositions including capsules and tablets have been developed to help maintain hydrated skin. Such products may for example contain vitamins, collagen, fatty acids, or plant extracts.

To improve the appearance of the skin, particularly the hydration of the skin, skincare products may be used which contain hyaluronic acid. Hyaluronic acid is a glycosaminoglycan (a mucopolysaccharide) which naturally occurs in human connective tissues, and forms part of the extracellular matrix. Hyaluronic acid is involved in the tissue repair process when skin is damaged or broken. Hyaluronic acid is a hygroscopic humectant, and can assist in maintaining hydrated skin. It is therefore used in topical cosmetic creams and orally administered tablets or capsules, as well as being a component of dermal fillers. WO 2013/040242 A2 discloses dermal filler compositions comprising hyaluronic acid, for the treatment of fine lines in facial skin. WO 2015/058734 A1 discloses a cosmetic composition comprising nanofibres containing hyaluronic acid and at least one carrier polymer. US 2019192553 A1 discloses a cosmetic composition comprising hyaluronic acid, dermatan sulfate, at least one omega-3 fatty acid, and at least one nucleotide. That composition can be orally administered as a food supplement.

Ceramides are a family of lipids. Ceramide lipid molecules are formed from sphingosine and a fatty acid (joined by an amide bond). Ceramides are found predominantly in eukaryotic cell membranes, and are components of sphingomyelin. Topically applied cosmetic formulations, particularly oily creams, can comprise ceramides as emollient skin conditioning agents, to improve the overall appearance of the skin. Ceramides may also be orally administered with a view to improving the appearance of the skin, particularly by improving the hydration of the skin. WO 2019/103194 A1 discloses an oil-in-water cosmetic composition for skin whitening or wrinkle reduction, comprising ceramide. KR 2019/0030190 A discloses a cosmetic composition which comprises ceramide particles dispersed in an amphiphilic solvent, for moisturising the skin. KR 101959278 B1 relates to a skin cosmetic composition comprising ceramide-coated beads, wherein the ceramides are extracted from microorganisms.

Hyaluronic acid and ceramides may optionally be combined in one cosmetic composition, for topical or oral administration. Delivering hyaluronic acid and ceramides in a single composition may enable the two components to act on the skin in synergy, by both attracting and sealing in water molecules. JP 2017/197470 A discloses a composition comprising ceramides and hyaluronic acid, which can be used to suppress water evaporation from the skin surface. That composition can be administered orally as a tablet or the like.

The present inventors have surprisingly found that an orally administered capsule, comprising both hyaluronic acid and wheat grain extract oil comprising phytoceramides, can comprise lower concentrations of ceramides than are known in the art, whilst still achieving a synergistic effect of hyaluronic acid and ceramides combined (that is, both attracting and sealing in water molecules) to improve the hydration and appearance of the skin. For example, the capsule of the present invention can help to maintain the hydration of the skin and reduce signs of aging such as fine lines and wrinkles. The present inventors have further surprisingly found that an orally administered capsule of the present invention can reduce and/or prevent the retinoid reaction.

### SUMMARY OF THE INVENTION

The present invention provides, according to a first aspect, a capsule for oral administration, comprising a filling having: from 30 to 50% by weight hyaluronic acid; and from 55 to 65% by weight of a wheat grain extract oil comprising phytoceramides; wherein the phytoceramides are present at from 0.1 to 5% by weight; wherein the % by weight of each component is measured relative to the total combined weight of the hyaluronic acid and wheat grain extract oil.

In one embodiment, the hyaluronic acid may be present in an amount of from 35 to 45% by weight. In another embodiment, the hyaluronic acid may be present in an amount of from 37.5 to 42.5% by weight. In a further embodiment, the hyaluronic acid may be present in an amount of 40% by weight. It will be appreciated that the % by weight of the hyaluronic acid present in the capsule filling is measured relative to the total combined weight of the hyaluronic acid and wheat grain extract oil.

In one embodiment, the hyaluronic acid comprises hyaluronic acid molecules having molecular weights in a range of from 50 to 2200 kDa.

In one embodiment, the wheat grain extract oil may be present in an amount of from 57.5 to 62.5% by weight. In a further embodiment, the wheat grain extract oil may be present in an amount of 60% by weight. It will be appreciated that the % by weight of the wheat grain extract oil present in the capsule filling is measured relative to the total combined weight of the hyaluronic acid and wheat grain extract oil.

In one embodiment, the phytoceramides may be present in an amount of from 0.5 to 3% by weight. In another embodiment, the phytoceramides may be present in an amount of from 1 to 2.5% by weight. In yet another embodiment, the phytoceramides may be present in an amount of from 1.2 to 2.4% by weight. It will be appreciated that the % by weight of the phytoceramides present in the capsule filling is measured relative to the total combined weight of the hyaluronic acid and wheat grain extract oil.

The capsule of the first aspect of the invention may have a total weight of from 1000 to 1500 mg. The total combined weight of the hyaluronic acid and wheat grain extract oil of the first aspect of the invention may be from 100 to 500 mg. In a preferred embodiment, the total combined weight of the hyaluronic acid and wheat grain extract oil is from 200 to 300 mg. In a particularly preferred embodiment, the total combined weight of the hyaluronic acid and wheat grain extract oil is from 225 to 275 mg.

The present invention provides, in a second aspect, a method of improving the hydration of human skin, comprising administering to a subject in need thereof the capsule of the first aspect of the invention. In some embodiments, the method may be a non-therapeutic method. Alternatively, the method may be a therapeutic method. In some embodiments, the method results in prevention or reduction of the severity of the retinoid reaction.

In one embodiment, the capsule is administered once daily, twice daily, three times daily, four times daily, or five times daily. Preferably, the capsule is administered twice daily. In one embodiment, the skin is the skin of one or more of the face, décolletage, neck and hands. In one embodiment, the method may further comprise administering to the subject a topical composition comprising one or more retinoids.

In a third aspect, the invention provides a kit comprising a capsule according to the first aspect of the invention, and a topical skin cream comprising one or more retinoids.

It will of course be appreciated that features described in relation to one aspect of the present invention may be incorporated into other aspects of the present invention.

### DETAILED DESCRIPTION

### Capsules for Oral Administration

The present invention provides, according to a first aspect, a capsule for oral administration, comprising a filling having: from 30 to 50% by weight hyaluronic acid; and from 55 to 65% by weight of a wheat grain extract oil comprising phytoceramides; wherein the phytoceramides are present in the capsule in an amount of from 0.1 to 5% by weight; wherein the % by weight of each component is measured relative to the total combined weight of the hyaluronic acid and wheat grain extract oil.

In one embodiment (for example, when the total combined weight of the hyaluronic acid and wheat grain extract oil is in a range of from 200 to 300 mg, such as from 225 to 275 mg) the hyaluronic acid (for example, hyaluronic acid comprising hyaluronic acid molecules having molecular weights in a range of from 50 to 2200 kDa) may be present in an amount of from 35 to 45% by weight. In another embodiment (for example, when the total combined weight of the hyaluronic acid and wheat grain extract oil is in a range of from 200 to 300 mg, such as from 225 to 275 mg) the hyaluronic acid (for example, hyaluronic acid comprising hyaluronic acid molecules having molecular weights in a range of from 50 to 2200 kDa) may be present in an amount of from 37.5 to 42.5% by weight. In a further embodiment (for example, when the total combined weight of the hyaluronic acid and wheat grain extract oil is in a range of from 200 to 300 mg, such as from 225 to 275 mg) the hyaluronic acid (for example, hyaluronic acid comprising hyaluronic acid molecules having molecular weights in a range of from 50 to 2200 kDa) may be present in an amount of 40% by weight. It will be appreciated that the % by weight of the hyaluronic acid present in the capsule filling is measured relative to the total combined weight of the hyaluronic acid and wheat grain extract oil.

In one embodiment (for example, when the total combined weight of the hyaluronic acid and wheat grain extract oil is in a range of from 200 to 300 mg, such as from 225 to 275 mg; and/or when the hyaluronic acid comprises hyaluronic acid molecules having molecular weights in a range of from 50 to 2200 kDa) the wheat grain extract oil may be present in an amount of from 57.5 to 62.5% by weight. In a further embodiment (for example, when the total combined weight of the hyaluronic acid and wheat grain extract oil is in a range of from 200 to 300 mg, such as from 225 to 275 mg; and/or when the hyaluronic acid comprises hyaluronic acid molecules having molecular weights in a range of from 50 to 2200 kDa) the wheat grain extract oil may be present in an amount of 60% by weight. It will be appreciated that the % by weight of the wheat grain extract oil present in the capsule filling is measured relative to the total combined weight of the hyaluronic acid and wheat grain extract oil.

In one embodiment (for example, when the total combined weight of the hyaluronic acid and wheat grain extract oil is in a range of from 200 to 300 mg, such as from 225 to 275 mg; and/or when the hyaluronic acid comprises hyaluronic acid molecules having molecular weights in a range of from 50 to 2200 kDa) the phytoceramides may be present in an amount of from 0.5 to 3% by weight. In another embodiment (for example, when the total combined weight of the hyaluronic acid and wheat grain extract oil is in a range of from 200 to 300 mg, such as from 225 to 275 mg; and/or when the hyaluronic acid comprises hyaluronic acid molecules having molecular weights in a range of from 50 to 2200 kDa) the phytoceramides may be present in an amount of from 1 to 2.5% by weight. In yet another embodiment (for example, when the total combined weight of the hyaluronic acid and wheat grain extract oil is in a range of from 200 to 300 mg, such as from 225 to 275 mg; and/or when the hyaluronic acid comprises hyaluronic acid molecules having molecular weights in a range of from 50 to 2200 kDa) the phytoceramides may be present in an amount of from 1.2 to 2.4% by weight. It will be appreciated that the % by weight of the phytoceramides present in the capsule filling is measured relative to the total combined weight of the hyaluronic acid and wheat grain extract oil

### Capsule

Capsules of the first aspect of the invention (for example, capsules wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) are formulated for oral administration. The capsule of the first aspect of the invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may have a total weight of from 1000 to 1500 mg; from 1000 to 1400 mg; from 1000 to 1300 mg; from 1000 to 1200 mg; from 1100 to 1200 mg; or from 1150 to 1200 mg. Preferably, the capsule of the first aspect of the invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) has a total weight of from 1150 to 1200 mg.

The total combined weight of the hyaluronic acid (for example, hyaluronic acid comprising hyaluronic acid molecules having molecular weights in a range of from 50 to 2200 kDa) and wheat grain extract oil may be in a range of from 100 to 500 mg; from 100 to 400 mg; from 100 to 300 mg; from 150 to 300 mg; from 200 to 300 mg; or from 225 to 275 mg. Preferably, the total combined weight of the hyaluronic acid (for example, hyaluronic acid comprising hyaluronic acid molecules having molecular weights in a range of from 50 to 2200 kDa) and wheat grain extract oil is in a range of from 225 to 275 mg.

### Capsule Shell

The capsule (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may be a soft gelatin capsule (commonly known as a softgel capsule) comprising a single-piece shell. The shell of a soft gelatin capsule comprises gelatin. The shell may comprise a plasticiser such as glycerol. The shell may comprise one or more colorants such as beta-carotene or caramel.

### Capsule Filling

The filling of the capsule (for example, a capsule wherein the total combined weight of the hyaluronic acid and wheat grain extract oil is in a range of from 200 to 300 mg, such as from 225 to 275 mg) comprises hyaluronic acid (for example, hyaluronic acid comprising hyaluronic acid molecules having molecular weights in a range of from 50 to 2200 kDa) and wheat grain extract oil, wherein the wheat grain extract oil comprises phytoceramides.

The filling of the capsule may comprise one or more lipid excipients such as soybean oil and rice bran oil. In certain preferred embodiments (for example, when the total combined weight of the hyaluronic acid and wheat grain extract oil is in a range of from 200 to 300 mg, such as from 225 to 275 mg; and/or when the hyaluronic acid comprises hyaluronic acid molecules having molecular weights in a range of from 50 to 2200 kDa) the lipid excipient is rice bran oil. The use of rice bran oil as an excipient in a softgel capsule filling is disclosed in PCT Publication No. WO 2008/139264 A2, the entirety of which is incorporated herein by reference.

The filling of the capsule (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may comprise a suspending agent such as beeswax. The use of beeswax as a suspending agent in a softgel capsule filling is disclosed in U.S. Pat. No. 6,955,820 B1, the entirety of which is incorporated herein by reference. Without wishing to be bound by theory, it is thought that the use of beeswax to thicken the suspension of hyaluronic acid in lipid wheat grain extract oil and rice bran oil helps to stabilise the suspension and prevent agglomeration of hyaluronic acid particles. Preventing the agglomeration of hyaluronic acid particles (for example, when the hyaluronic acid comprises hyaluronic acid molecules having molecular weights in a range of from 50 to 2200 kDa) may ensure the softgel capsule does not deteriorate upon storage.

The filling of the capsule (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may comprise one or more botanical extracts with antioxidant properties, such as a green tea leaf extract, a grape seed extract, a sea buckthorn extract, a pine bark extract, astaxanthin, lycopene, resveratrol, or a rosemary leaf extract, preferably a rosemary leaf extract. The botanical extract, such as rosemary leaf extract, may be provided in a sunflower oil solution.

The filling of the capsule (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may comprise one or more nutritional supplements comprising one or more elements such as calcium, phosphorus, potassium, sodium, magnesium, sulfur, iron, chlorine, cobalt, copper, zinc, molybdenum, iodine, selenium and manganese. The filling of the capsule (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may comprise one or more nutritional supplements such vitamin A, vitamin C, vitamin D, vitamin E, omega 3, and omega 6. In certain preferred embodiments, the nutritional supplement comprises manganese. The manganese may be present as manganese gluconate. The manganese may be present in an amount of from 0 to 1% by weight relative to the total combined weight of the hyaluronic acid and wheat grain extract oil. The manganese may be present in an amount of from 0.05 to 0.5% by weight relative to the total combined weight of the hyaluronic acid and wheat grain extract oil. The manganese may be present in an amount of from 0.05 to 0.2% by weight relative to the total combined weight of the hyaluronic acid and wheat grain extract oil. The manganese may be present in an amount of 0.16% by weight relative to the total combined weight of the hyaluronic acid and wheat grain extract oil.

### Method of Preparing the Capsule

Methods of preparing a filled soft gelatin capsule (commonly known as a softgel capsule) are known in the art and are disclosed, for example, in Soft Gelatin Capsules (Softgels), Raj, A, PharmaTutor, 2015, 3(10):16-18, the entirety of which is incorporated herein by reference; and US Patent Publication No. U.S. Pat. No. 5,200,191 A, the entirety of which is likewise incorporated herein by reference.

A method of preparing the capsule of the first aspect of the invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may be provided as follows.

The method may comprise preparing the soft gelatin capsule shell. The components of the shell of the soft gelatin capsule may be mixed and melted to form a molten composition, which is then extruded to form a film.

The method may comprise preparing the filling of the soft gelatin capsule. The components of the capsule filling, including hyaluronic acid (for example, hyaluronic acid comprising hyaluronic acid molecules having molecular weights in a range of from 50 to 2200 kDa) in solid powder form and wheat grain extract oil in liquid form, may be mixed. The capsule filling may be sufficiently well-mixed to form a homogeneous paste, which comprises a stable suspension of hyaluronic acid in a lipid phase comprising wheat grain extract oil and, optionally, a lipid excipient such as rice bran oil and/or a suspending agent such as beeswax. In certain embodiments (for example, when the total combined weight of the hyaluronic acid and wheat grain extract oil is in a range of from 200 to 300 mg, such as from 225 to 275 mg; and/or when the hyaluronic acid comprises hyaluronic acid molecules having molecular weights in a range of from 50 to 2200 kDa) the components of the capsule filling do not separate upon storage for up to one month, two months, three months, four months, five months, six months, one year, or two years. Without wishing to be bound by theory, it is thought that the use of beeswax to thicken the suspension of hyaluronic acid (for example, hyaluronic acid comprising hyaluronic acid molecules having molecular weights in a range of from 50 to 2200 kDa) in the lipid phase helps to stabilise the suspension and prevent agglomeration of hyaluronic acid particles. Preventing the agglomeration of hyaluronic acid particles (for example, when the hyaluronic acid molecules have molecular weights in a range of from 50 to 2200 kDa) may ensure the softgel capsule does not deteriorate upon storage. For example, the softgel capsule may not deteriorate upon storage for up to one month, two months, three months, four months, five months, six months, one year, or two years. In certain embodiments (for example, when the total combined weight of the hyaluronic acid and wheat grain extract oil is in a range of from 200 to 300 mg, such as from 225 to 275 mg; and/or when the hyaluronic acid comprises hyaluronic acid molecules having molecular weights in a range of from 50 to 2200 kDa) the beeswax may be present at levels of between 0.01% to 10%, for example between 0.1% and 6%, for example between 0.5% and 6%, for example between 1% and 6% by weight relative to the total combined weight of the hyaluronic acid and wheat grain extract oil.

Preparing the filling of the soft gelatin capsule may comprise mixing the components of the capsule filling using a turbulent flow mixer, for example a turbulent flow mixer obtained from Dinex, Fynsvej 39, DK-5500 Middelfart, Denmark.

Preparing the filling of the soft gelatin capsule may additionally or alternatively comprise milling the components of the capsule filling, to ensure that single particles of hyaluronic acid are homogeneously distributed throughout the wheat grain extract oil lipid phase (for example, when the hyaluronic acid molecules have molecular weights in a range of from 50 to 2200 kDa). The milling may be carried out using a mixer mill, for example a mixer mill obtained from Retsch GmbH, Retsch-Allee 1-5, 42781 Haan, Germany.

Preparing the filling of the soft gelatin capsule may additionally or alternatively comprise mixing the components of the capsule filling using a homogeniser, for example a homogeniser obtained from Ystral, The Scientific Instrument Centre Ltd, 13 East Links, Tollgate, Chandlers Ford, Hampshire SO53 3TG, United Kingdom.

The method of preparing the capsule may comprise encapsulating the filling of the soft gelatin capsule in the shell of the soft gelatin capsule. Typically, encapsulation is carried out by injecting the capsule filling into a soft gelatin shell pocket formed between two ribbons of soft gelatin shell film disposed on twin rotating dies. As the ribbons meet, the capsule filling is precisely injected in a pocket formed between them, to encapsulate the capsule filling in a soft gelatin shell. The twin rotating dies continuously rotate, thereby sealing and dispensing the capsule product.

The capsule (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may be dried and packaged, for example in a jar or a blister pack. The packaging may optionally include a leaflet or instructions describing, for example, the recommended dosage regime. For example, the leaflet or instructions may recite one or more of the dosage regimes described herein. The leaflet or instructions may disclose the use of the products and/or methods of the invention, for example their use to increase the level of hydration of the skin. The leaflet or instructions may disclose the use of the products and/or methods of the invention to prevent or reduce signs of aging. Additionally or alternatively, the leaflet or instructions may disclose the use of the products and/or methods of the invention to prevent or decrease the severity of the retinoid reaction in a subject who will undergo, is undergoing, or has undergone a regime of applying a topical skin cream comprising one or more retinoids.

### Hyaluronic Acid

Hyaluronic acid (also known as hyaluronan; conjugate base hyaluronate) is a non-sulfated glycosaminoglycan (a mucopolysaccharide). Hyaluronic acid is a linear polymer and can have a molecular weight of up to about 7000 kilodaltons (kDa). In some embodiments (for example, when the total combined weight of the hyaluronic acid and wheat grain extract oil is in a range of from 200 to 300 mg, such as from 225 to 275 mg) the hyaluronic acid of the present invention may comprise hyaluronic acid molecules having molecular weights in a range of from 10 to 5000 kDa; from 10 to 4500 kDa; from 10 to 4000 kDa; from 10 to 3500 kDa; from 20 to 3500 kDa; from 30 to 3000 kDa; from 50 to 3000 kDa; from 50 to 2500 kDa; from 50 to 2300 kDa; or from 50 to 2200 kDa. In some embodiments (for example, when the total combined weight of the hyaluronic acid and wheat grain extract oil is in a range of from 200 to 300 mg, such as from 225 to 275 mg) the hyaluronic acid of the present invention comprises hyaluronic acid molecules having molecular weights in a range of from from 30 to 220 kDa; from 40 to 210 kDa; or from 50 to 200 kDa. In some embodiments (for example, when the total combined weight of the hyaluronic acid and wheat grain extract oil is in a range of from 200 to 300 mg, such as from 225 to 275 mg) the hyaluronic acid of the present invention comprises hyaluronic acid molecules having molecular weights in a range of from 100 to 700 kDa; from 150 to 650 kDa; or from 200 to 600 kDa. In some embodiments (for example, when the total combined weight of the hyaluronic acid and wheat grain extract oil is in a range of from 200 to 300 mg, such as from 225 to 275 mg) the hyaluronic acid of the present invention comprises hyaluronic acid molecules having molecular weights in a range of from 800 to 2000 kDa; from 900 to 1900 kDa; or from 1000 to 1800 kDa. In some embodiments (for example, when the total combined weight of the hyaluronic acid and wheat grain extract oil is in a range of from 200 to 300 mg, such as from 225 to 275 mg) the hyaluronic acid of the present invention comprises hyaluronic acid molecules having molecular weights in a range of from 1600 to 2400 kDa; from 1700 to 2300 kDa; or from 1800 to 2200 kDa. In certain preferred embodiments (for example, when the total combined weight of the hyaluronic acid and wheat grain extract oil is in a range of from 200 to 300 mg, such as from 225 to 275 mg) the hyaluronic acid of the present invention comprises hyaluronic acid molecules having molecular weights in a range of from 50 to 3000 kDa. In particularly preferred embodiments (for example, when the total combined weight of the hyaluronic acid and wheat grain extract oil is in a range of from 200 to 300 mg, such as from 225 to 275 mg) the hyaluronic acid of the present invention comprises hyaluronic acid molecules having molecular weights in a range of from 50 to 2200 kDa.

Hyaluronic acid naturally occurs in human connective, epithelial and neural tissues, and is a component of the extracellular matrix. Hyaluronic acid is involved in physiological functions including lubrication and water homeostasis, and is involved in the tissue repair process when skin is damaged or broken.

Hyaluronic acid is a hygroscopic humectant. Without wishing to be bound by theory, it is thought that hyaluronic acid molecules (for example, hyaluronic acid molecules having molecular weights in a range of from 50 to 2200 kDa) present in the extracellular matrix of the stratum corneum can attract and retain water molecules, thereby increasing the level of hydration of human skin.

Hyaluronic acid for use in the capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may be provided in solid powder form. Hyaluronic acid may be obtained as ExceptionHYAL™ Star from Rolemi HPC, 24-26 Via Celeste Milani, 21040 Origgio (VA) Italy.

### Wheat Grain Extract Oil

The wheat grain extract oil of the first aspect of the invention comprises phytoceramides. Wheat grain extract oil may be obtained as Lipowheat™ from Robertet Health and Beauty, 10 Avenue Yves-Emmanuel Baudoin, 06130, Grasse, France.

Wheat grain extract oil for use in the capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may preferably be provided in liquid form. Lipid ceramides are readily soluble in the liquid wheat grain extract oil.

### Phytoceramides

Phytoceramides are ceramides derived from plants. Wheat grain extract oil derived from wheat grain may comprise phytoceramides. The wheat grain extract oil of the first aspect of the invention comprises phytoceramides.

Ceramides are waxy lipid molecules. A ceramide molecule comprises sphingosine and a fatty acid, linked by an amide bond.

In eukaryotic cells, ceramides are components of the sphingomyelin of the cell membrane lipid bilayer.

The term “ceramides”, as used herein, encompasses all sources of ceramides and ceramide derivatives, including, for example, glucosylceramides.

Without wishing to be bound by theory, it is thought that ceramides have emollient properties. Ceramides are able to coat the surface of skin cells, thereby providing an occlusive lipid coating on the stratum corneum, blocking water evaporation from the skin surface. This increases the level of hydration of the skin.

### Product Packaging

The capsules of the first aspect of the invention (for example, capsules wherein the total combined weight of the hyaluronic acid and wheat grain extract oil in each capsule is in a range of from 200 to 300 mg, such as from 225 to 275 mg; and/or wherein the hyaluronic acid comprises hyaluronic acid molecules having molecular weights in a range of from 50 to 2200 kDa) may be provided in suitable packaging. Suitable packaging may comprise, for example, a jar or a blister pack. The packaging may optionally include a leaflet or instructions describing, for example, the recommended dosage regime. For example, the leaflet or instructions may recite one or more of the dosage regimes described herein. The leaflet or instructions may disclose the use of the products and/or methods of the invention, for example their use to increase the level of hydration of the skin. The leaflet or instructions may disclose the use of the products and/or methods of the invention to prevent or reduce signs of aging. Additionally or alternatively, the leaflet or instructions may disclose the use of the products and/or methods of the invention to prevent or decrease the severity of the retinoid reaction in a subject who will undergo, is undergoing, or has undergone a regime of applying a topical skin cream comprising one or more retinoids (for example vitamin A).

### Method of Improving the Hydration of Human Skin

The present invention provides, in a second aspect, a method of improving the hydration of human skin, comprising administering to a subject in need thereof one or more capsule(s) having any of the features described above (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa).

The method of the second aspect of the invention may be a non-therapeutic method. Alternatively, the method may be a therapeutic method. In an embodiment, the capsule (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) is administered once daily, twice daily, three times daily, four times daily, or five times daily. Preferably, the capsule (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) is administered twice daily. In an embodiment, the skin is one or more of the skin of the face, décolletage, neck and hands. In certain preferred embodiments (for example, when the total combined weight of the hyaluronic acid and wheat grain extract oil is in a range of from 200 to 300 mg, such as from 225 to 275 mg; and/or when the hyaluronic acid comprises hyaluronic acid molecules having molecular weights in a range of from 50 to 2200 kDa) the skin is the skin of the face, for example the skin of the cheek. In an embodiment, the method may further comprise administering to the subject a topical composition comprising one or more retinoids. In some embodiments, the method results in prevention or reduction of the severity of the retinoid reaction.

### Hydration of Human Skin

Without wishing to be bound by theory, it is thought that hyaluronic acid (for example, hyaluronic acid comprising hyaluronic acid molecules having molecular weights in a range of from 50 to 2200 kDa) increases the hydration of the human skin by acting as a hygroscopic humectant. Hyaluronic acid (for example, hyaluronic acid comprising hyaluronic acid molecules having molecular weights in a range of from 50 to 2200 kDa) can attract and retain water molecules in the extracellular matrix of the stratum corneum. It is thought that ceramides act as emollients, coating the skin cells of the stratum corneum and thereby providing an occlusive coating which blocks water loss by evaporation. When both hyaluronic acid and ceramides are provided to human skin (in particular to the stratum corneum) the two can act in synergy to increase the level of hydration of the skin, by both attracting and sealing in water molecules.

The capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) provides both hyaluronic acid and ceramides to the skin. The capsule of the present invention can therefore increase the level of hydration of human skin. More particularly, the capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) can increase the level of hydration of the stratum corneum of human skin.

A conventional method of assessing the level of hydration of the stratum corneum comprises measurement of the total electrical resistance (impedance, Z) of the skin to an alternating current of frequency F. The total impedance (Z) depends on the resistance (R) and capacitance (C) of the skin. The impedance of the stratum corneum varies with the level of hydration of the skin. Thus, capacitive devices can be used to estimate the level of hydration of the skin. The level of hydration of the skin can be measured by a capacitive sensor such as a Courage+Khazaka Dual 580 Multi Probe Adapter System fitted with a Corneometer™ CM 825 probe, obtained from Courage±Khazaka electronic GmbH, Mathias-Brüggen-Str. 91, 50829 Köln, Germany.

The level of hydration of human skin may additionally or alternatively be determined by monitoring transepidermal water loss (TEWL). TEWL may be measured by measuring the water flow density in grams per second per square metre (g/sm²). For example, a conventional open-chamber TEWL sensor, comprising two moisture sensors positioned in a measuring head, can determine TEWL. Each of the two moisture sensors measures the partial pressure of the water vapour immediately adjacent to the skin surface. The gradient in partial pressure between the two sensors is directly proportional to the rate of evaporation from the skin. Given the length and diameter of the open-chamber measuring head, the TEWL value can be calculated. TEWL may be measured by an open-chamber TEWL sensor such as a Courage+Khazaka Dual 580 Multi Probe Adapter System fitted with a Tewameter™ TM 300 probe, obtained from Courage±Khazaka electronic GmbH, Mathias-Brüggen-Str. 91, 50829 Köln, Germany.

The products of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) and the methods of the present invention may increase the level of hydration of the skin, for example as when measured by a capacitive device. The products of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) and the methods of the present invention may increase the level of hydration of the skin by 1 to 80%; 1 to 70%; by 1 to 60%; %; by 1 to 50%; by 10 to 70%; by 10 to 65%; by 10 to 60%; by 10 to 50%; by 15 to 70%; by 15 to 65%; by 15 to 60%; by 15 to 55%; by 15 to 50%; by 20 to 70%; by 20 to 65%; by 20 to 60%; by 20 to 55%; by 20 to 50%; by 25 to 70%; by 25 to 65%; by 25 to 60%; by 25 to 55%; by 25 to 50%; by 25 to 45%; by 25 to 40%; by 30 to 70%, by 30 to 65%, by 30 to 60%, by 30 to 55%, by 30 to 50%, by 30 to 45%, or by 30 to 40%, for example as when measured by a capacitive device. In certain preferred embodiments (for example, when the total combined weight of the hyaluronic acid and wheat grain extract oil is in a range of from 200 to 300 mg, such as from 225 to 275 mg; and/or when the hyaluronic acid comprises hyaluronic acid molecules having molecular weights in a range of from 50 to 2200 kDa) the products and methods of the present invention may increase the level of hydration of the skin by 10 to 70%, for example as when measured by a capacitive device. In certain preferred embodiments (for example, when the total combined weight of the hyaluronic acid and wheat grain extract oil is in a range of from 200 to 300 mg, such as from 225 to 275 mg; and/or when the hyaluronic acid comprises hyaluronic acid molecules having molecular weights in a range of from 50 to 2200 kDa) the products and methods of the present invention may increase the level of hydration of the skin of the cheek by at least 30%, at least 40%, at least 50% or at least 60%, for example as when measured by a capacitive device. It will be appreciated that the percentage increase in the level of hydration of the skin of a subject is measured relative to the level of hydration of the skin of the subject prior to treatment with the products and/or methods of the present invention.

The products of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) and the methods of the present invention may decrease TEWL, for example as when measured by an open-chamber TEWL sensor. The products of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) and the methods of the present invention may decrease TEWL by 1 to 50%; by 1 to 45%; by 1 to 40%; by 1 to 35%; by 1 to 30%; by 1 to 25%; by 1 to 20%; by 5 to 50%; by 5 to 45%; by 5 to 40%; by 5 to 35%; by 5 to 30%; by 5 to 25%; by 5 to 20%; by 10 to 50%; by 10 to 45%; by 10 to 40%; by 10 to 35%; by 10 to 30%; by 10 to 25%; or by 10 to 20%, for example as when measured by an open-chamber TEWL sensor. In certain preferred embodiments (for example, when the total combined weight of the hyaluronic acid and wheat grain extract oil is in a range of from 200 to 300 mg, such as from 225 to 275 mg; and/or when the hyaluronic acid comprises hyaluronic acid molecules having molecular weights in a range of from 50 to 2200 kDa) the products and methods of the present invention may decrease TEWL of the skin by 10 to 30%, for example as when measured by an open-chamber TEWL sensor. In certain preferred embodiments (for example, when the total combined weight of the hyaluronic acid and wheat grain extract oil is in a range of from 200 to 300 mg, such as from 225 to 275 mg; and/or when the hyaluronic acid comprises hyaluronic acid molecules having molecular weights in a range of from 50 to 2200 kDa) the products and methods of the present invention may decrease the TEWL of the skin of the cheek by at least 5%, at least 10%, at least 50%, or at least 20%, for example as when measured by an open-chamber TEWL sensor. It will be appreciated that the percentage decrease in the TEWL of the skin of a subject is measured relative to TEWL of the skin of the subject prior to treatment with the products and/or methods of the present invention.

### Appearance of Human Skin

Without wishing to be bound by theory, it is thought that increasing the level of hydration of the stratum corneum of the skin may lead to a decrease in the appearance of wrinkles and/or fine lines in the skin. The capsules of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) can increase the level of hydration of the skin, thereby leading to a decrease in the appearance of wrinkles and/or fine lines in the skin.

The depth and/or width of wrinkles in the skin may be measured in μm. The depth and/or width of wrinkles in the skin may be measured using a high-resolution camera adapted to measure the depth and/or width of wrinkles in the skin. For example, the depth and/or width of wrinkles in the skin may be measured using a Courage+Khazaka Visoscan™ VC98 obtained from Courage Khazaka electronic GmbH, Mathias-Brüggen-Str. 91, 50829 Köln, Germany.

The products of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) and the methods of the present invention may decrease the depth of wrinkles in the skin, for example as when measured using a high-resolution camera adapted to measure the depth and/or width of wrinkles in the skin. The products of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) and the methods of the present invention may decrease the depth of wrinkles in the skin by 1 to 50%; by 1 to 45%; by 1 to 40%; by 1 to 35%; by 1 to 30%; by 1 to 25%; by 1 to 20%; by 5 to 50%; by 5 to 45%; by 5 to 40%; by 5 to 35%; by 5 to 30%; or by 10 to 30%, for example as when measured using a high-resolution camera adapted to measure the depth of wrinkles in the skin. In certain preferred embodiments (for example, when the total combined weight of the hyaluronic acid and wheat grain extract oil is in a range of from 200 to 300 mg, such as from 225 to 275 mg; and/or when the hyaluronic acid comprises hyaluronic acid molecules having molecular weights in a range of from 50 to 2200 kDa) the products and methods of the present invention may decrease the depth of wrinkles in the skin by 10 to 30%, for example as when measured using a high-resolution camera adapted to measure the depth of wrinkles in the skin. It will be appreciated that the percentage decrease in the depth of wrinkles in the skin of a subject is measured relative to the depth of wrinkles in the skin of the subject immediately prior to treatment with the products and/or methods of the present invention.

The products of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) and the methods of the present invention may decrease the width of wrinkles in the skin, for example as when measured using a high-resolution camera adapted to measure the width of wrinkles in the skin. The products of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) and the methods of the present invention may decrease the width of wrinkles in the skin by 0.1 to 50%; by 0.1 to 45%; by 0.1 to 40%; by 0.1 to 35%; by 0.1 to 30%; by 0.1 to 25%; by 0.1 to 20%; by 5 to 50%; by 5 to 45%; by 5 to 40%; by 5 to 35%; by 5 to 30%; or by 10 to 30%, for example as when measured using a high-resolution camera adapted to measure the width of wrinkles in the skin. In certain preferred embodiments (for example, when the total combined weight of the hyaluronic acid and wheat grain extract oil is in a range of from 200 to 300 mg, such as from 225 to 275 mg; and/or when the hyaluronic acid comprises hyaluronic acid molecules having molecular weights in a range of from 50 to 2200 kDa) the products and methods of the present invention may decrease the width of wrinkles in the skin by 10 to 30%, for example as when measured using a high-resolution camera adapted to measure the width of wrinkles in the skin. It will be appreciated that the percentage decrease in the width of wrinkles in the skin of a subject is measured relative to the width of wrinkles in the skin of the subject immediately prior to treatment with the products and/or methods of the present invention.

### Dosage Regimes

The effective amount of hyaluronic acid and ceramides administered in a capsule of the present invention will be dependent on factors including, but not limited to, the subject being treated (including their age, weight, sex and physiological condition) the pre-treatment level of hydration of the subject's skin, the desired post-treatment level of hydration of the subject's skin, and whether the subject will undergo, is undergoing, or has undergone a treatment regime of administering a topical cosmetic cream comprising one or more retinoids (for example vitamin A).

The capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may be administered once daily, twice daily, three times daily, four times daily, or five times daily. Preferably, the capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) is administered twice daily.

Alternatively, the capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may be administered once every two, three or four days.

The capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may be administered in a dosage regime sufficient to provide a dose of from 1 to 2 mg hyaluronic acid per kg body weight per day; from 1.2 to 1.8 mg hyaluronic acid per kg body weight per day; from 1.3 to 1.7 mg hyaluronic acid per kg body weight per day; or from 1.4 to 1.6 mg hyaluronic acid per kg body weight per day. Such a dosage regime may be particularly suitable when the capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) is administered once daily.

The capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may be administered in a dosage regime sufficient to provide a dose of from 2 to 4 mg hyaluronic acid per kg body weight per day; from 2.4 to 3.6 mg hyaluronic acid per kg body weight per day; from 2.6 to 3.4 mg hyaluronic acid per kg body weight per day; or from 2.8 to 3.2 mg hyaluronic acid per kg body weight per day. Such a dosage regime may be particularly suitable when the capsule of the present invention is administered twice daily. Preferably, the capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) is administered twice daily.

The capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may be administered in a dosage regime sufficient to provide a dose of from 3 to 6 mg hyaluronic acid per kg body weight per day; from 3.6 to 5.4 mg hyaluronic acid per kg body weight per day; from 3.9 to 5.1 mg hyaluronic acid per kg body weight per day; or from 4.2 to 4.8 mg hyaluronic acid per kg body weight per day. Such a dosage regime may be particularly suitable when the capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) is administered three times daily.

The capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may be administered in a dosage regime sufficient to provide a dose of from 4 to 8 mg hyaluronic acid per kg body weight per day; from 4.8 to 7.2 mg hyaluronic acid per kg body weight per day; from 5.2 to 6.8 mg hyaluronic acid per kg body weight per day; or from 5.6 to 6.4 mg hyaluronic acid per kg body weight per day. Such a dosage regime may be particularly suitable when the capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) is administered four times daily.

The capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may be administered in a dosage regime sufficient to provide a dose of from 5 to 10 mg hyaluronic acid per kg body weight per day; from 6 to 9 mg hyaluronic acid per kg body weight per day; from 6.5 to 8.5 mg hyaluronic acid per kg body weight per day; or from 7 to 8 mg hyaluronic acid per kg body weight per day. Such a dosage regime may be particularly suitable when the capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) is administered five times daily.

The capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may be administered in a dosage regime sufficient to provide a dose of from 0.015 to 0.15 mg phytoceramides per kg body weight per day; from 0.03 to 0.12 mg phytoceramides per kg body weight per day; from 0.03 to 0.11 mg phytoceramides per kg body weight per day; or from 0.046 to 0.092 mg per kg body weight per day. Such a dosage regime may be particularly suitable when the capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) is administered once daily.

The capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may be administered in a dosage regime sufficient to provide a dose of from 0.03 to 0.3 mg phytoceramides per kg body weight per day; from 0.06 to 0.24 mg phytoceramides per kg body weight per day; from 0.06 to 0.22 mg phytoceramides per kg body weight per day; or from 0.092 to 0.18 mg per kg body weight per day. Such a dosage regime may be particularly suitable when the capsule of the present invention is administered twice daily. Preferably, the capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) is administered twice daily.

The capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may be administered in a dosage regime sufficient to provide a dose of from 0.045 to 0.45 mg phytoceramides per kg body weight per day; from 0.09 to 0.36 mg phytoceramides per kg body weight per day; from 0.09 to 0.33 mg phytoceramides per kg body weight per day; or from 0.14 to 0.28 mg per kg body weight per day. Such a dosage regime may be particularly suitable when the capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) is administered three times daily.

The capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may be administered in a dosage regime sufficient to provide a dose of from 0.06 to 0.6 mg phytoceramides per kg body weight per day; from 0.12 to 0.48 mg phytoceramides per kg body weight per day; from 0.12 to 0.44 mg phytoceramides per kg body weight per day; or from 0.18 to 0.37 mg per kg body weight per day. Such a dosage regime may be particularly suitable when the capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) is administered four times daily.

The capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may be administered in a dosage regime sufficient to provide a dose of from 0.075 to 0.75 mg phytoceramides per kg body weight per day; from 0.15 to 0.6 mg phytoceramides per kg body weight per day; from 0.15 to 0.55 mg phytoceramides per kg body weight per day; or from 0.23 to 0.46 mg per kg body weight per day. Such a dosage regime may be particularly suitable when the capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) is administered five times daily. It will be understood that the phytoceramides of the capsule of the present invention are ceramides provided in a wheat grain extract oil derived from wheat grain. The term “ceramides”, as used herein, encompasses all sources of ceramides and ceramide derivatives, including, for example, glucosylceramides.

### The Retinoid Reaction

Retinoids are a class of compounds comprising vitamin A and its derivatives. Retinoid molecules consist of a polar end group, a polyene side chain and a cyclic end group. Retinoids include, for example, retinol, retinal, tretinoin (retinoic acid), isotretinoin, alitretinoin, etretinate (and its metabolite acitretin), adapalene, bexarotene, and tazarotene.

Retinoids (for example vitamin A) are commonly administered in skincare products, to improve the appearance of the skin. In particular, retinoids (for example vitamin A) may be topically applied to human skin to reduce or prevent acne, diminish the appearance of fine lines, and decrease hyperpigmentation.

However, subjects administering topically applied compositions comprising retinoids (for example vitamin A) may experience adverse effects. This phenomenon is commonly known as the retinoid reaction. Adverse effects may include, for example, inflammation, increased skin sensitivity, irritation of the skin, redness of the skin, peeling of the skin, and a decrease in the level of hydration of the skin.

Subjects may be particularly prone to experiencing the retinoid reaction during the winter months, when human skin is commonly less hydrated and when the transepidermal water loss (TEWL) of the stratum corneum is higher, compared to the same human skin during the summer months.

An adverse effect of the retinoid reaction is an increase in the redness of the skin. The measurement of skin tone can provide an indication of the severity of the retinoid reaction in a subject. For example, skin tone can be measured using a colorimeter such as a Chroma Meter CR-400 colorimeter, obtainable from Konica Minolta Sensing Singapore Pte Ltd, 9 Webster Court, Westbrook Crescent, Gemini Business Park, Warrington, WAS 8WD, United Kingdom. A subject experiencing the retinoid reaction may experience an increase in the redness of the skin, for example the skin of one or more of the face, neck, décolletage and hands (relative to the normal redness of their skin, or relative to the skin of a control group not experiencing the retinoid reaction), for example when measured by a colorimeter. The percentage increase in the redness of the subject's skin (measured relative to the normal redness of their skin, or relative to the skin of a control group not experiencing the retinoid reaction) may be a percentage increase of from 1 to 100%; from 1 to 90%; from 1 to 80%; from 1 to 70%; from 1 to 60%; from 1 to 50%; from 1 to 40%; from 1 to 30%; from 1 to 20%; from 1 to 10%; or from 1 to 5%, for example as when measured by a colorimeter.

Products according to the first aspect of the invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) can be particularly useful in reducing or preventing the adverse effects of the retinoid reaction. Methods according to the second aspect of the invention (for example, methods wherein the capsule has a total combined weight of hyaluronic acid and wheat grain extract oil in a range of from 200 to 300 mg, such as from 225 to 275 mg; and/or wherein the hyaluronic acid of the capsule comprises hyaluronic acid molecules having molecular weights in a range of from 50 to 2200 kDa) can also be particularly useful in reducing or preventing the adverse effects of the retinoid reaction.

### Kits

The present invention provides, in a third aspect, a kit comprising a capsule having any of the features described above (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) and a topical skin cream comprising one or more retinoids (for example vitamin A).

It is thought that hyaluronic acid (for example, hyaluronic acid comprising hyaluronic acid molecules having molecular weights in a range of from 50 to 2200 kDa) and ceramides can both increase the hydration of the skin. Delivering hyaluronic acid and ceramides to the skin in a single composition in the capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) can enable both components to increase the level of hydration of the skin in synergy, by both attracting and sealing in water molecules.

Hydrated skin is less susceptible to the adverse effects of the retinoid reaction. The capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) can therefore prevent or reduce the adverse effects of the retinoid reaction in a subject, wherein the subject is also administered a topical skin cream comprising one or more retinoids (for example vitamin A).

The capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may be administered to a subject before the subject has begun a regime of applying a topical skin cream comprising one or more retinoids (for example vitamin A). Additionally or alternatively, the capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may be administered to a subject while the subject is undergoing a regime of applying a topical skin cream comprising one or more retinoids (for example vitamin A). Additionally or alternatively, the capsule of the present invention (for example, a capsule wherein the total combined weight of the hyaluronic acid and wheat grain extract oil is in a range of from 200 to 300 mg, such as from 225 to 275 mg; and/or wherein the hyaluronic acid comprises hyaluronic acid molecules having molecular weights in a range of from 50 to 2200 kDa) may be administered to a subject after the subject has ended a regime of applying a topical skin cream comprising one or more retinoids (for example vitamin A).

### Topical Skin Cream

The topical skin cream of the kit of the invention comprises one or more retinoids (for example vitamin A). The one or more retinoids (for example vitamin A) may be present in an amount sufficient to cause the adverse effects of the retinoid reaction in a subject. The topical skin cream may be a cosmetic cream. The topical skin cream may be a moisturising cream.

The one or more retinoids (for example vitamin A) may be present in the topical skin cream in an amount sufficient to cause the adverse effects of the retinoid reaction in a subject, which may be from 0.01 to 2% by weight; 0.01 to 1% by weight; 0.01 to 0.5% by weight; from 0.01 to 0.45% by weight; from 0.01 to 0.4% by weight; from 0.01 to 0.35% by weight; from 0.01 to 0.3% by weight; from 0.01 to 0.25% by weight; from 0.01 to 0.2% by weight; from 001 to 0.15% by weight; from 001 to 0.1% by weight; or from 001 to 0.05% by weight; wherein the % by weight is measured relative to the total weight of the topical skin cream.

The one or more retinoids (for example vitamin A) may be present in the topical skin cream in an amount sufficient to cause the adverse effects of the retinoid reaction in a subject when the topical skin cream is administered at an amount of from 0.1 to 5 ml/cm² of the skin; 0.1 to 4 ml/cm² of the skin; 0.1 to 3 ml/cm² of the skin; 0.1 to 2 ml/cm² of the skin; 0.1 to 1 ml/cm² of the skin; at 0.1 to 0.9 ml/cm² of the skin; at 0.1 to 0.8 ml/cm² of the skin; at 0.1 to 0.7 ml/cm² of the skin; at 0.1 to 0.6 ml/cm² of the skin; at 0.1 to 0.5 ml/cm² of the skin; at 0.1 to 0.3 ml/cm² of the skin; at 0.1 to 0.2 ml/cm² of the skin; at 0.001 to 0.1 ml/cm² of the skin; at 0.001 to 0.075 ml/cm² of the skin; at 0.001 to 0.05 ml/cm² of the skin; at 0.001 to 0.025 ml/cm² of the skin or 0.001 to 0.01 ml/cm² of the skin.

The topical skin cream comprising one or more retinoids (for example vitamin A) may be administered to the skin, for example the topical skin cream may be administered to the skin in any of the amounts described immediately above, and left in contact with the skin for a period of time. The topical skin cream may be left in contact with the skin for a period of time sufficient to cause the adverse effects of the retinoid reaction in a subject. For example, in certain embodiments the topical skin cream may be administered to the skin and left in contact with the skin for a period of 5 minutes or more; 10 minutes or more; 15 minutes or more; 20 minutes or more; 1 hour or more; 2 hours or more; 3 hours or more; 4 hours or more; 5 hours or more; 6 hours or more; 12 hours or more; 24 hours or more; or 36 hours or more.

Any suitable method of applying the topical skin cream to the skin in need thereof may be used in accordance with the present invention. For example, the topical skin cream may be applied to the skin of the face, neck or hands, or a combination of two or more thereof. In certain preferred embodiments, the topical skin cream is applied to the skin of the face.

The topical skin cream may be applied directly from a package to the skin; by hand to the skin; or from a substrate such as a wipe to the skin; or by a combination of two or more of such methods of application.

### EXAMPLES

### Example 1

A softgel capsule was prepared having the shell composition of Table 1a and the filling composition of Table 1b.

TABLE 1a
Shell Composition of Softgel Capsule

| Shell component | Quantity in milligrams (mg) |
|---|---|
| Fish Gelatin | 223.38 |
| Glycerol | 143.56 |
| Betacarotene (30% by weight in | 8.88 |
| sunflower oil) | |
| Caramel colour E150a | 8.88 |

The total weight of the shell of Table 1a is 385.00 mg.

TABLE 1b
Filling Composition of Softgel Capsule

| Filling component | Quantity in milligrams (mg) |
|---|---|
| Rice bran oil | 500.00 |
| Wheat grain extract oil | 150.00 |
| Hyaluronic acid (consisting of hyaluronic | 100.00 |
| acid molecules having molecular weights | |
| in a range of from 50 to 2200 kDa) | |
| Beeswax | 30.36 |
| Manganese gluconate | 3.64 |

The total weight of the filling of Table 1b is 784.00 mg.

### Example 2

The softgel capsule of Example 1 was administered twice daily to 12 subjects for 12 weeks. The age of the subjects ranged from 30 to 61 years. The elasticity, hydration and transepidermal water loss (TEWL) of the skin surrounding the eye, the skin of the cheek, the skin of the neck, and the skin of the forearm of each subject was measured on the first day of the 12-week study and on the last day of the 12-week study. The depth and width of wrinkles in those areas of the subjects' skin was also measured on the first day of the 12-week study and on the last day of the 12-week study.

A Courage+Khazaka Dual 580 Multi Probe Adapter System fitted with a Cutometer™ MPA 580 probe was used to measure the elasticity of the subjects' skin.

A Courage+Khazaka Dual 580 Multi Probe Adapter System fitted with a Corneometer™ CM 825 probe was used to measure the surface hydration of the subjects' skin.

A Courage+Khazaka Dual 580 Multi Probe Adapter System fitted with a Tewameter™ TM 300 probe was used to measure the TEWL of the subjects' skin.

A Courage+Khazaka Visoscan™ VC98 was used to measure the depth and width of wrinkles in the subjects' skin.

The average percentage change in each of the parameters measured from the first day of the twelve week study to the last day of the twelve week study was calculated. The results are shown in Table 2.

TABLE 2

| | | Average percentage change over the |
|---|---|---|
| Parameter | Skin region | course of the 12-week study |
| Elasticity | Eye | −7% |
| | Cheek | −6% |
| | Neck | −3% |
| | Arm | −2% |
| Wrinkle width | Eye | −20% |
| | Cheek | +16% |
| | Neck | −19% |
| | Arm | +3% |
| Wrinkle depth | Eye | −28% |
| | Cheek | +18% |
| | Neck | −25% |
| | Arm | +9% |
| Skin surface | Eye | +31% |
| hydration | Cheek | +64% |
| | Neck | +38% |
| | Arm | +11% |
| TEWL | Eye | +18% |
| | Cheek | −20% |
| | Neck | −17% |
| | Arm | −21% |

Those results show a decrease over the course of the 12-week study in the width and depth of wrinkles in the skin surrounding the eye and in the skin of the neck. This supports the conclusion that administering the capsule of the present invention to a subject may lead to a decrease in the appearance of fine lines and/or wrinkles in the subject's skin.

The results also demonstrate an increase in skin surface hydration and a corresponding decrease in transepidermal water loss for each of the skin of the cheek, the skin of the neck and the skin of the arm. This supports the conclusion that administering the capsule of the present invention to a subject may lead to an increase in the level of hydration of the subject's skin, in particular the level of hydration of the stratum corneum. Hydrated skin is less susceptible to the adverse effects of the retinoid reaction. The capsule of the present invention may therefore prevent or reduce the adverse effects of the retinoid reaction in a subject, wherein the subject is also administered a topical skin cream comprising one or more retinoids.

### Comparative Example 3

A cachet containing 200 mg hyaluronic acid (HA) was administered orally, once daily, to 30 subjects for 28 days. The hyaluronic acid administered in this manner consisted of hyaluronic acid molecules having molecular weights in a range of from 50 to 2200 kDa.

The hydration, TEWL and wrinkle depth of the skin of the cheek of each subject was measured on the first day of the 28-day study and on the last day of the 28-day study.

A Courage+Khazaka Dual 580 Multi Probe Adapter System fitted with a Corneometer™ CM 825 probe was used to measure the surface hydration of the subjects' skin.

A Courage+Khazaka Dual 580 Multi Probe Adapter System fitted with a Tewameter™ TM 300 probe was used to measure the TEWL of the subjects' skin.

A PRIMOS 3D™ optical skin measurement device was used to measure the depth and width of wrinkles in the subjects' skin.

The average percentage change in each of the parameters measured from the first day of the 28-day study to the last day of the 28-day study was calculated. The results are shown in Table 3.

TABLE 3

| | Average percentage change over the |
|---|---|
| Parameter | course of the 28-day study |
| Wrinkle depth | −18.8% |
| Skin surface hydration | +10.6% |
| TEWL | −3.9% |

### Comparative Example 4

350 mg wheat grain extract oil (comprising 2 to 4% phytoceramides by weight, relative to the total weight of the wheat grain extract oil) was administered orally, once daily, to 25 subjects for three months.

The hydration of the skin of the cheek of each subject was measured on the first day of the three-month study and on the last day of the three-month study.

The average percentage change in the hydration of the skin of the cheek from the first day of the three-month study to the last day of the three-month study was calculated and was found to be +26.0%.

The increase in the level of hydration of the skin of the cheek resulting from administration of the softgel capsule of Example 1 (see Table 2 above) demonstrates a greater than additive effect (+64.0%) compared to the sum of the increases seen for HA alone (+10.6%) and phytoceramides alone (+26.0%). This suggests that, when administered in combination, HA and phytoceramides act in synergy to increase the level of hydration of the skin.

TABLE 4

| | Average percentage change in the |
|---|---|
| | hydration of the skin surface of |
| Example | the cheek over the course of the study |
| Example 2 | +64.0% |
| (HA and ceramides) | |
| Comparative Example 3 | +10.6% |
| (HA alone) | |
| Comparative Example 4 | +26.0% |
| (ceramides alone) | |

### Example 5

The softgel capsule of Example 1 was administered twice daily to 12 subjects for 12 weeks. The age of the subjects ranged from 30 to 61 years.

Prior to the experiment, of the 12 subjects tested, 5 of them self-reported that they typically suffered from the retinoid reaction every winter from the topical application of cosmetic preparations containing vitamin A. Following consumptions of the capsules, these 5 subjects self-reported a significant reduction in the severity of the retinoid reaction.

1. A capsule for oral administration, comprising a filling having:
from 30 to 50% by weight hyaluronic acid; and
from 55 to 65% by weight of a wheat grain extract oil comprising phytoceramides;
wherein the phytoceramides are present in the capsule in an amount of from 0.1 to 5% by weight;
wherein the % by weight of each component is measured relative to the total combined weight of the hyaluronic acid and wheat grain extract oil.

2. The capsule of claim 1, wherein the hyaluronic acid comprises hyaluronic acid molecules having molecular weights in a range of from 50 to 2200 kDa.

3. The capsule of claim 1, wherein the total combined weight of the hyaluronic acid and the wheat grain extract oil is from 100 to 500 mg.

4. The capsule of claim 3, wherein the total combined weight of the hyaluronic acid and the wheat grain extract oil is from 200 to 300 mg.

5. The capsule of claim 4, wherein the total combined weight of the hyaluronic acid and the wheat grain extract oil is from 225 to 275 mg.

6. The capsule of claim 1, wherein the phytoceramides are present in an amount of from 0.5 to 3% by weight.

7. The capsule of claim 1, wherein the hyaluronic acid is present in an amount of from 35 to 45% by weight.

8. The capsule of claim 1, wherein the phytoceramides are present in an amount of from 1 to 2.5% by weight.

9. The capsule of claim 1, wherein the hyaluronic acid is present in an amount of from 37.5 to 42.5% by weight.

10. The capsule of claim 1, wherein the wheat grain extract oil is present in an amount of from 57.5 to 62.5% by weight.

11. The capsule of claim 1, wherein the phytoceramides are present in an amount of from 1.2 to 2.4% by weight.

12. The capsule of claim 1, wherein the total weight of the capsule is from 1000 to 1500 mg.

13. A method of improving the hydration of human skin, comprising administering to a subject in need thereof the capsule of claim 1.

14. The method of claim 13, wherein the method is non-therapeutic.

15. The method of claim 13, wherein the capsule is administered once daily, twice daily, three times daily, four times daily, or five times daily.

16. The method of claim 15, wherein the capsule is administered twice daily.

17. The method of claim 13, wherein the skin comprises one or more of: the skin of the face, décolletage, neck and hands.

18. The method of claim 13, further comprising administering to the subject a topical composition comprising one or more retinoids (for example vitamin A).

19. A kit comprising: one or more capsule(s) according to claim 1; and a topical skin cream comprising one or more retinoids (for example vitamin A).
